(19) United States
(12) Patent Application Publication
(10) Pub. No.: US 2005/0181457 A1
(43) Pub. Date: Aug. 18, 2005

(54) FcyrIII signaling inhibitor assay

(76) Inventors: Thomas Baumruker, Moedling (AT); Eva Erika Prieschl, Wien (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/513,805

(22) PCT Filed: May 16, 2003
(86) PCT No.: PCT/EP03/05175

(30) Foreign Application Priority Data
May 17, 2002 (US) 60381544
May 28, 2002 (US) 60383652

(51) Int. Cl. A61K 39/395; G01N 33/53; G01N 33/567

(52) U.S. Cl. 435/7.2; 424/131.1

(57) ABSTRACT

The present invention provides an assay and its use for identifying agents that inhibit the FcγRIII signaling.

The present invention relates to an assay and its use for identifying an agent that inhibits the FcγRIII signaling using a stably transfected cell line.

Recent data with complement- and FcγR-knock out mice resulted in a paradigm shift in the understanding of the etiology of certain autoimmune diseases such as e.g. Lupus, Glomerulonephritis. While up to now many of the autoimmune diseases were regarded to be in part or fully dependent on the complement system, these recent findings provided conclusive evidence that unwanted triggering/signaling initiated at FcγRs and in particular the FcγRIII are causally involved in the pathogenesis. Therefore the signaling cascades and their participating molecules may comprise novel targets for pharmacological approaches in the prevention of autoimmune diseases, such as Lupus and Glomerulonephritis, and other diseases.

Fc receptors are expressed on a variety of cell types including macrophages, mast cells, neutrophils and natural killer cells. Triggering by cross-linking of Fc receptors with antibodies or antibody-antigen complexes mediates a wide variety of cellular effector functions, commonly providing a link between the innate and adaptive immune system. The pathological importance of these systems was demonstrated recently by the unexpected finding that mice deficient in Fc receptors were unable to mount inflammatory responses when immunoglobulins (IgG) are bound to their cognate antigens (see e.g. Ravetch, J. V. 1997, Fc receptors, Curr. Opin. Immunol. 9:121-125). In marked contrast animals deficient in complement components had a normal inflammatory response to these experimentally induced (cytotoxic) antibodies and IgG-antigen complexes, abolishing an old immunological dogma about the pathogenesis of many autoimmune diseases.

Pathogenic IgG antibodies arise in autoimmune diseases, including systemic lupus erythematosus, idiopathic thrombocytopenia purpura, immune hemolytic anemia, rheumatoid arthritis and allergic asthma and are furthermore (alone or in combination with further factors) considered as the pathogenic triggers for such diseases. In addition, it was shown experimentally that mice deficient in IgG Fc receptors are protected from disease in models of systemic lupus, glomerulonephritis, idiopathic thrombocytopenia purpura, hemolytic anemia and allergic asthma. It is now firmly established that the FcγRIII receptor in particular plays an essential role in such disease processes. Such observations form the basis for an entirely new approach to the treatment of intractable antibody mediated autoimmune diseases all of them being currently treated with IVIG (IntraVenous Immunoglobulin G). While detailed understanding of the mechanism of action of IVIG is still under active investigation, it is conceivable that the high amounts of IVIG may interact with cellular FcRs and specifically with the FcγRIII thereby preventing activation of the cells. The specificity of the FcγRIII pathway in coupling cytotoxic and immune complex antibodies to effector responses strongly suggests that an FcγRIII signaling inhibitor will have a similar activity comparable with IVIG with all the advantages of a low molecular weight compound.

We have now surprisingly found and validated a stable, genetically modified cell line which is especially useful for high throughput screening of inhibitors (esp. low molecular weight inhibitors) of the FcγRIII signaling after triggering with an IgG stimulus, e.g. a stimulus with IgG complexes.

The present invention provides in one aspect an assay for identifying an agent that inhibits the FcγIII signaling comprising:
a) providing a cell line stably transfected with a construct comprising an inducible specific promoter, corresponding 3′ regulatory gene sequences and a reporter gene, operatively linked to each other,
b) providing a candidate compound,
c) contacting the cell line of a) with an immunoglobulin G complex for a sufficient period of time to obtain a stimulated cell line,
d) adding the candidate compound of b) to the cell line of a) or to the stimulated cell line of c) simultaneously with the immunoglobulin G complex or shortly thereafter, or not adding the candidate compound of b),
e) detecting the expression of the reporter gene and/or its product in the presence and in the absence of a candidate compound and determining whether there is a difference in the expressed amount of the reporter gene and/or its product in the absence and in the presence of a candidate compound,
f) choosing an agent from said candidate compound detected in e), e.g. for use as a pharmaceutical.

Appropriate cell lines in an assay of the present invention include any cell line where the FcγRIII signaling pathway can be triggered via an immunoglobulin G complex. Such stably transfected cell lines include e.g. a DC18 mouse cell line stably transfected with an inducible specific promoter, corresponding 3′ regulatory gene sequences and a reporter gene, operatively linked to each other.

An appropriate inducible specific promoter includes any DNA regulatory sequence that responds positively to a triggering of the FcγRIII and which is linked to an appropriate detectable system, e.g. a reporter gene. The inducible promoter is e.g. an hTNFα promoter, the corresponding 3′ regulatory gene sequence is e.g. from the hTNFα gene.

An appropriate reporter gene includes genes, the amount of expression of which are detectable, e.g. by a method as conventional, e.g. luciferase gene, secreted alkaline phosphatase or fluorescence proteins, like e.g. GFP (=green fluorescence protein).

The cell line used in an assay according to the present invention is characterized in that it expresses FcγRIII at mRNA level and strongly responds with TNF-α secretion when stimulated with an appropriate substance (stimulus), e.g. with IgG complexes.

In one embodiment of the present invention the candidate compound is not added to the cell line stimulated or not stimulated with the immunoglobulin G complex.

In another embodiment of the present invention the screening assay can be carried out in that the stably transfected cell line is pre-stimulated with e.g. an IgG-complex and the candidate compound is added after a certain time period, e.g. shortly thereafter, so that the candidate compound still can elicit its inhibitory function. The expression of the reporter gene and/or its product in the presence and in the absence of the candidate compound is detected as described earlier, the difference determined and an agent may be chosen.

Appropriate candidate compounds include compound(s)(libraries) from which its influence on the Fc-receptor can be determined. Compound (libraries) include for example oligopeptides, polypeptides, proteins, antibodies, mimetics, small molecules, e.g. low molecular weight compounds (LMW's).

Since the cell lines used in the present invention strongly respond with TNF-α secretion when stimulated with appropriate substances, e.g. with IgG complexes, or to a reduction of TNF-α secretion in case the candidate compound has an inhibitory action on the FcγRIII signaling pathway (=agent), an appropriate agent can be identified in using these cells in an assay according to the present invention.

When TNF-α secretion is reduced also less reporter gene and/or gene product is expressed. The specific decrease in the expression of the reporter gene in the cell line contacted with the candidate compound in the given concentration in comparison with the expression of the reporter gene in the stimulated cell not contacted with the candidate compound indicates that and to what degree the candidate compound inhibits FcγRIII signaling.

The expressed amount of the reporter gene and/or its product is detected. A “gene product” is e.g. the expressed protein, polypeptide or a fragment thereof.

An agent is a compound which influences (inhibits) the expression of the reporter gene and/or its products detected/determined in step e). An agent identified by an assay of the present invention is therein defined as “an agent of the present invention”.

An agent of the present invention is one of the chosen candidate compounds and may include oligopeptides, polypeptides, proteins, antibodies, mimetics, small molecules, e.g. low molecular weight compounds (LMW's).

Preferably an agent of the present is a candidate compound which is able to decrease the amount of expression of the reporter gene e.g. by 35% or more in comparison with the amount of reporter gene expressed by the cell line in the absence of a candidate compound.

The stimulation or triggering of the cell line may be of importance for the sensitivity of the assay (ratio stimulated vs. non-stimulated). In a preferred embodiment an IgG-F(ab′)₂ complex and more preferred an IgG2a-F(ab′)₂ complex may be used as the stimulating immunoglobulin G (IgG)-antigen complex.

Further parameters like e.g. time for contacting, stimulation, number of cells required, solvent used may be optimized according, e.g. analogously, to a method as conventional.

In another aspect the present invention provides a kit comprising
a) a cell line stably transfected with a construct comprising an inducible specific promoter, corresponding 3′ regulatory gene sequences and a reporter gene, operatively linked to each other,
b) an immunoglobulin G-antigen complex and
c) detection means.

Components a) and b) may be provided in appropriate solvents and/or buffer solutions. Appropriate detection means include conventional means, e.g. include fluorescence spectroscopy or ELISA techniques as conventional.

In another aspect the present invention provides an agent which inhibits FcγRIII signaling and which is identified by the assay according to the present invention for use as a pharmaceutical.

An agent of the present invention may exhibit pharmacological activity and is therefore useful as a pharmaceutical, e.g. against autoimmune related diseases including but not limited to ITP (=idiopathic thrombocytopenia purpura), systemic lupus erythematosus, immune hemolytic anemia or rheumatoid arthritis or allergic diseases like e.g. allergic asthma or type I allergies.

An agent of the present invention may show therapeutic activity against autoimmune related diseases and allergic diseases.

An agent of the present invention for treatment includes one or more, preferably one, agent of the present invention, e.g. a combination of two or more agents of the present invention.

In another aspect the present invention provides an agent for use as a pharmaceutical.

In another aspect the present invention provides the use of an agent of the present invention for the manufacture of a medicament, e.g. a pharmaceutical composition, for the treatment of autoimmune related diseases including but not limited to ITP (=idiopathic thrombocytopenia purpura), systemic lupus erythematosus, immune hemolytic anemia or rheumatoid arthritis or allergic diseases like e.g. allergic asthma or type I allergies.

In a further aspect the present invention provides a method of treatment of autoimmune related diseases and allergic diseases, which treatment comprises administering to a subject in need of such treatment an effective amount of a compound of the present invention; e.g. in the form of a pharmaceutical composition.

Treatment includes treatment and prophylaxis.

For such treatment, the appropriate dosage will, of course, vary depending upon, for example, the chemical nature and the pharmacokinetic data of an agent of the present invention employed, the individual host, the mode of administration and the nature and severity of the conditions being treated. However, in general, for satisfactory results in larger mammals, for example humans, an indicated daily dosage is in the range from about 0.01 g to about 1.0 g, of an agent of the present invention; conveniently administered, for example, in divided doses up to four times a day.

An agent of the present invention may be administered by any conventional route, for example enterally, e.g. including nasal, buccal, rectal, oral administration; parenterally, e.g. including intravenous, intramuscular, subcutanous administration; or topically; e.g. including epicutaneous, intranasal, intratracheal administration; e.g. in form of coated or uncoated tablets, capsules, injectable solutions or suspensions, e.g. in the form of ampoules, vials, in the form of creams, gels, pastes, inhaler powder, foams, tinctures, lip sticks, drops, sprays, or in the form of suppositories.

An agent of the present invention may be administered in the form of a pharmaceutically acceptable salt, e.g. an acid addition salt or metal salt; or in free form; optionally in the form of a solvate. An agent of the present invention in the form of a salt may exhibit the same order of activity as an agent of the present invention in free form; optionally in the form of a solvate.

An agent of the present invention may be used for pharmaceutical treatment according to the present invention alone, or in combination with one or more other pharmaceutically active agents.

Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

In another aspect the present invention provides a pharmaceutical composition comprising an agent of the present invention in association with at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In another aspect the present invention provides a pharmaceutical composition according to the present invention, further comprising another pharmaceutically active agent.

Such compositions may be manufactured according, e.g. analogously to a method as conventional, e.g. by mixing, granulating, coating, dissolving or lyophilizing processes. Unit dosage forms may contain, for example, from about 0.5 mg to about 1000 mg, such as 1 mg to about 500 mg.

### DESCRIPTION OF THE FIGURES

FIG. 1: Stimulation of DC18 C10 cells by various immunoglobulin complexes (including IgG isotypes); nst=non stimulated;

FIG. 2: Time kinetics for stably transfected DC18 C10 cells stimulated with IgG2a complexes

FIG. 3: Application of various inhibitors in the given concentration (x-axis) on IgG2a-complex stimulated DC18 C10 cells

FIG. 4: Titration of the cell number (indicated at the x-axis) to provide for a robust readout

In the following examples all temperatures are given in degree Celcius and are uncorrected.

The following ABBREVIATIONS are used:

CsA=Cyclosporin A
DNP-BSA=dinitrophenyl-bovine serum albumin
FCS=fetal calf serum
HEPES=(N-(2-Hydroxyethyl)piperazine-N-(2-ethanesulfonic acid))
mGM-CSF=mouse granulocyte monocyte-colony stimulating factor
PMA=phorbol-12-Myristate-13-acetate
RPMI=cell culture medium named after the Roswell Park Memorial Institute
TNP=trinitrophenyl

### EXAMPLE 1

Production of Transfected Cell Lines and Cell Cultivation:

The DC18 cell line is characterized in e.g. Elbe A. et al., J. Immunol.153:2878-2889, 1994; Prieschl E. E. et al., J. Immunol.157:2645-2653, 1996.

In short, cells are isolated/grown out from mouse fetal skin cell suspensions, containing dermal and epidermal cells after enrichment for viable cells using a Lymphocyte-M gradient. Cells are seeded at a density of 2×10⁵ per well in 96-well round bottom tissue culture plates using RPMI 1640. To the medium 10% FCS, 25 mM HEPES, 50 μg/ml of gentamycin, 2 mM L-glutamine, 0.1 mM non-essential amino acids, 1 mM sodium pyruvate, 50 μM β-mercaptoethanol and an antibiotio-antimycotic solution are added. The culture medium is additionally supplemented with mGM-CSF (100 U/ml).

DC18 clone—genetically engineered cells are generated as follows:

A hTNFαpromoter—luciferase—hTNFα 3′ construct is used for generating stable cell lines.

Cells are transfected by electroporation at optimised conditions (200V, 960 pF) with a TNFα promoter—luciferase—TNFα 3′ end construct and plated in 10 ml of medium immediately after electroporation and purified by Lymphoprep the next day. 1×10⁴ viable cells per well are plated in 96 well microtiter plates in 100 μl of medium each plus 250 μg/ml of geneticin (G418). Two days later 100 μl of fresh medium are added. Every second day 100 μl of medium are removed and substituted by 100 μl of fresh medium. Stable clones are selected by conventional cell culture methods and single cell cloning. This results in 7 outgrowing clones of which 4 respond with the induction of luciferase upon stimulation with IgG complexes to a variable degree (between 6 to 10 fold). The best responder is subjected to one round of further limited dilution cloning (single cell cloning) and named DC18 clone10 (=DC18 C10). This clone is selected for all further studies.

Since the stimulation of the assay is via IgG, it is of vital importance that the cells are cultured in low IgG medium to prevent prior stimulation during the culturing period.

### EXAMPLE 2

Stimulation Conditions and Readout:

IgG subtypes are provided from Pharmingen/Becton Dickinson, DNP-BSA from Calbiochem and rabbit anti mouse Ig F(ab′)₂ fragment from Jackson Laboratories.

1×10⁴ cells are seeded in 50 μl medium per well (Optiplate, Packard). Incubation with candidate compounds is done for 45 minutes prior to stimulation with 5 μg/ml of murine IgG of different subtypes complexed either with 10 μg/ml of DNP-BSA or 30 μg/ml of rabbit anti mouse Ig F(ab′)₂ fragments. Stimulation is carried on for further 4 hours. Subsequently 13 μl of a 5 times concentrated reporter lysis buffer (Promega) are added and cells are lysed by freeze-(−80°) thaw cycle. For the readout 50 μl of luciferase substrate (Promega) are added and luciferase activity is measured in a MicroBetaJet (Wallac).

First experiments with this clone prove that complex formation by IgG plus F(ab′)₂ is superior to complex formation by IgG plus antigen (DNP-BSA) with respect to maximally induced values of luciferase and TNFα in the supernatant as well as overall reproducibility.

Furthermore, monomeric IgG does not stimulate TNFα induction, in agreement with no detectable expression of the high affinity receptor for IgG, the FcγRI. Therefore, in all further experiments a stimulation by IgG plus F(ab′)₂ is performed.

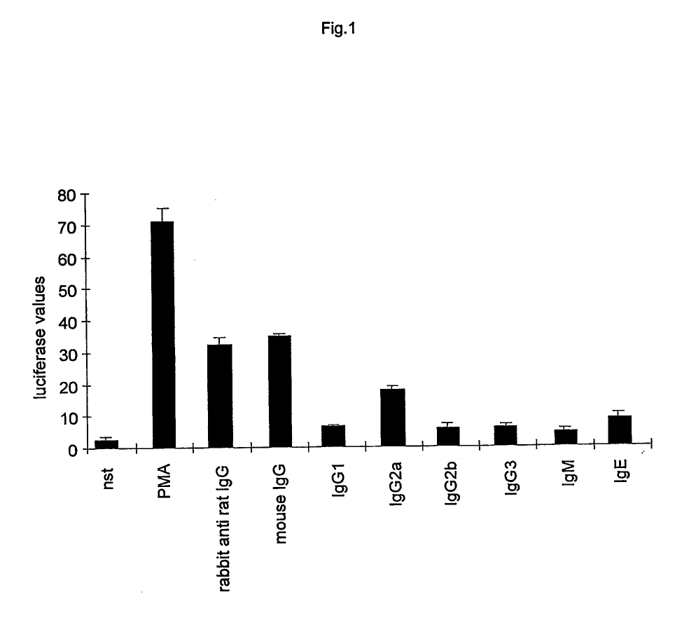

As the FcγRIII in mouse and humans interacts differently with different IgG isotypes, stimulation of DC18 C10 cells by complexes of IgG isotypes are tested instead of whole IgG, namely IgG1, IgG2a, IgG2b and IgG3 complexes. As a comparison, a PMA stimulation as well as a stimulation with IgM and IgE is done side by side. As shown in FIG. 1, the best stimulation (for IgGs) is achieved with IgG2a, which is used in all further studies as the triggering immunoglobulin.

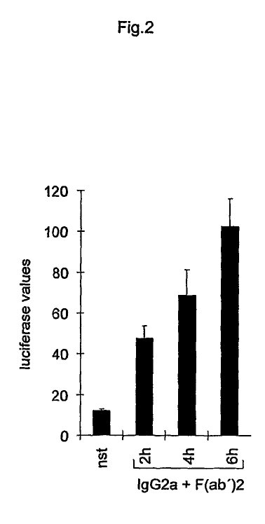

To determine optimal readout conditions, time kinetics of stimulation are performed over an 6 hours range. As can be seen in FIG. 2, maximal stimulation is achieved at the 6 hours time-point. As the ratio non-stimulated to stimulated, however, is already more than 10-fold at 4 hours, such stimulation for the shorter time period of 4 hours is preferred for practical reasons (to complete the assay in one day including pre-incubation with drugs and measurement) for all further studies,.

### EXAMPLE 3

Screening for Prototype Inhibitors:

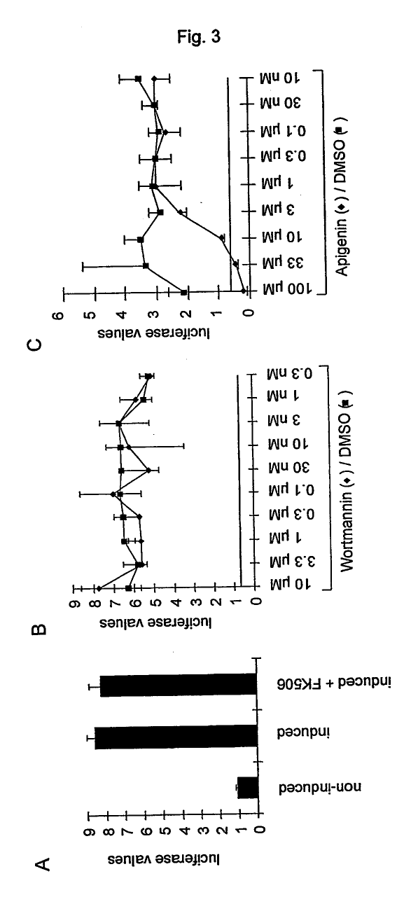

In search for a prototype inhibitor (positive control) several candidate compounds known to inhibit certain signaling pathways are exploited. Neither CsA (Sandimmune; data not shown), FK506 (FIG. 3A) or Wortmannin (a PI3-kinase inhibitor; FIG. 3B) inhibit the readout significantly at IC₅₀ concentrations. Quite in contrast, Apigenin, a commercially available erk1,2/jnk1,2 inhibitor, inhibits IgG2a complexes-provoked luciferase induction at the described IC₅₀ concentration (see FIG. 3C).

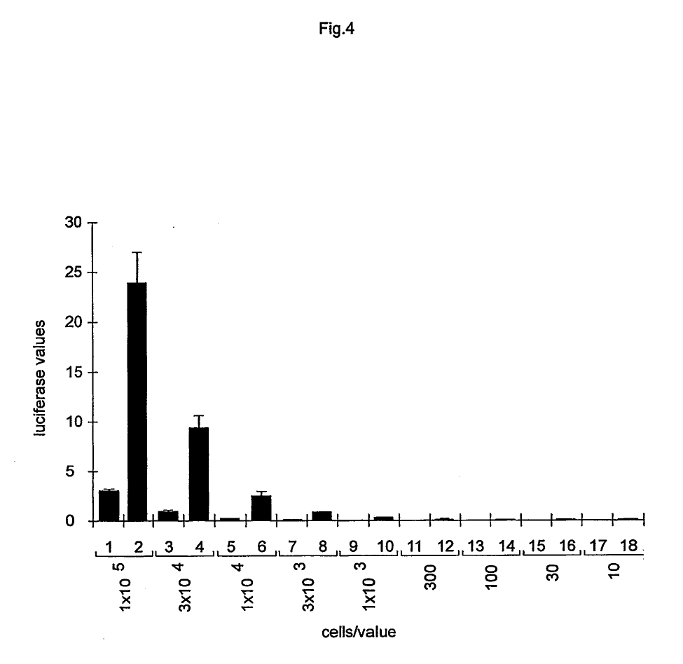

To optimize for high throughput screening a titration of the number of cells required in order to obtain a suitable/significant luciferase readout is carried out. As can be seen in FIG. 4, 10⁴ DC18 C10 cells per well in 96 well microtiter plate are sufficient for a robust and easy-to-detect readout.

### EXAMPLE 4

Establishment of an Assay Protocol:

An assay protocol is established as follows.
1.) 10⁴ DC18 C10 cells per well are used in 45 μl of medium (Packard Optiplate) and 5 μl of compounds in solution/solvent control are added,
2.) Incubation is carried out for 45 minutes at 37° in a humidified incubator by 5% CO₂,
3.) Stimulation is carried out with 5 μg/ml of mouse IgG2a anti-TNP plus 30 μg/ml of rabbit anti-mouse Ig(Fab′)₂ fragment,
4.) Incubation is carried out for 4 hours at 37° in a humidified incubator by 5% CO₂,
5.) 13 μl of a 5 times concentrated lysis buffer (Promega) are added,
6.) the composition is frozen for 1 hour at −80° , cells are thawed again so that they are lysed (lysis by freeze thaw cycle),
7.) 50 μl of luciferase reagent (Promega) are added to the lysed cells and with 5 seconds lag time, 5 seconds measuring time in a Wallac MicroBetaJet measurements of luciferase activity are done.

The cut off point for the definition of a hit is set at 35% inhibition at 5 μM concentration of substance, but can be chosen also differently, e.g. at 60% inhibition.

The whole procedure including handling the cells and measuring can be done in a six to seven hours time-span, making the assay easy to handle for high throughput screening. Using this assay protocol we also validated the assay by checking for high/low values, solvent sensitivity and performed a number of test racks with various substances.

1. An assay for identifying an agent that inhibits the FcγRIII signaling comprising:
a) providing a cell line stably transfected with a construct comprising an inducible specific promoter, corresponding 3′ regulatory gene sequences and a reporter gene, operatively linked to each other,
b) providing a candidate compound,
c) contacting the cell line of a) with an immunoglobulin G complex for a sufficient period of time to obtain a stimulated cell line,
d) adding the candidate compound of b) to the cell line of a) or to the stimulated cell line of c) simultaneously with the immunoglobulin G complex or shortly thereafter, or not adding the candidate compound of b),
e) detecting the expression of the reporter gene and/or its product in the presence and in the absence of a candidate compound and determining whether there is a difference in the expressed amount of the reporter gene and/or its product in the absence and in the presence of a candidate compound,
f) choosing an agent from said candidate compound detected in e), e.g. for use as a pharmaceutical.

2. Assay according to claim 1, characterized in that the immunoglobulin G (IgG)-antigen complex is an IgG-F(ab′)₂ complex.

3. A kit comprising
a) a cell line stably transfected with a construct comprising an inducible specific promoter, corresponding 3′ regulatory gene sequences and a reporter gene, operatively linked to each other,
b) an immunoglobulin G-antigen complex, and
c) detection means.

4. An agent identified by an assay according to claim 1.

5. An agent of claim 4 for use as a pharmaceutical.

6. The use of an agent of claim 4 for the manufacture of a medicament for the treatment of an autoimmune related disease or an allergic disease.

7. A pharmaceutical composition comprising an agent identified by an assay according to claim 1 in association with at least one pharmaceutical excipient.

8. A method of treatment of an autoimmune related disease or an allergic disease, which treatment comprises administering to a subject in need of such treatment an effective amount of an agent identified by an assay according to claim 1 or a pharmaceutical composition comprising an agent identified by an assay according to claim 1 in association with at least one pharmaceutical excipient.

9. A method according to claim 8 characterized in that the autoimmune related disease is idiopathic thrombocytopenia purpura, systemic lupus erythematosus, immune hemolytic anemia or rheumatid arthritis and the allergic disease is allergic asthma.
